(19) United States
(12) Patent Application Publication
(10) Pub. No.: US 2026/0199615 A1
(43) Pub. Date: Jul. 16, 2026

(54) METERED DOSE INHALER

(71) Applicant: Kindeva Drug Delivery L.P., Woodbury, MN (US)

(72) Inventors: Lee Hodges, Clitheroe (GB); Kerry Ward, Fortrose (GB); Steve Howgill, Leicester (GB); John Bunting, Castle Donington (GB)

(21) Appl. No.: 19/136,574

(22) PCT Filed: Nov. 30, 2023
(86) PCT No.: PCT/US2023/081933

§ 371 Date: Jun. 6, 2025

Related U.S. Application Data
(60) Provisional application No. 63/430,761, filed on Dec. 7, 2022.

(51) Int. Cl. A61M 15/00; A61M 11/02

(52) U.S. Cl. CPC A61M 15/009; A61M 11/02; A61M 15/002

(57) ABSTRACT

Various embodiments of a metered dose inhaler are disclosed. The inhaler includes a container and a metering valve connected to the container. The metering valve includes a valve housing having first and second ends and a metering valve stem that extends through the first end, a metering chamber, and the second end of the valve housing. A first portion of the metering valve stem is disposed through a diaphragm opening of a diaphragm that is disposed at least partially within the first end of the housing and forms a dynamic seal with the diaphragm, and a second portion of the metering valve stem is disposed through a tank seal opening of a tank seal that is disposed at least partially within the second end of the housing and forms a dynamic seal with the tank seal. At least one of the diaphragm or tank seal includes a polytetrafluoroethylene material.

This application claims the benefit of U.S. Provisional Application No. 63/430,761, filed Dec. 7, 2022, the disclosure of which is incorporated by reference herein in its entirety.

### BACKGROUND

Delivery of aerosolized medicament to the respiratory tract for the treatment of respiratory and other diseases can be done using, by way of example, pressurized metered dose inhalers (pMDI), dry powder inhalers (DPI), or nebulizers. pMDIs are familiar to many patients who suffer from asthma or from chronic obstructive pulmonary disease (COPD). pMDI devices can include an aluminum container, sealed with a metering valve, which contains medicament formulation. Generally, the medicament formulation is a solution and/or suspension of one or more medicinal compounds in a liquefied hydrofluoroalkane (HFA) propellant.

In pulmonary pMDIs, the sealed container can be provided to the patient in an actuator—a generally L-shaped plastic part that includes a generally vertical tube that surrounds the container plus a generally horizontal tube that forms a patient portion (e.g., a mouthpiece or nosepiece) that can define an inspiration (or inhalation) orifice.

The container typically includes a metering valve that is crimped onto an appropriately-sized metal can. The metal can is typically made of aluminum and has a wall thickness of approximately 0.5 mm. The container holds a formulation typically including liquid propellant(s), drug(s), co-solvent(s) and excipient(s). To prevent loss of the formulation, (primarily the liquid propellant), the metering valve contains rubber components that form seals. Historically, the propellants in most pMDIs had been chlorofluorocarbons (CFCs).

Environmental concerns during the 1990s have, however, led to the replacement of CFCs with hydrofluoroalkanes (HFAs) as the most commonly used propellant in pMDIs. Although HFAs do not cause ozone depletion, they do have a stated high global warming potential (GWP), which is a measurement of the future radiative effect of an emission of a substance relative to that of the same amount of carbon dioxide (CO₂). The two HFA propellants most commonly used in pMDIs are HFA134a (CF₃CH₂F) and HFA 227 (CF₃CHFCHF₃) having stated 100-year GWP values of 1300 to 1430 and 3220 to 3350, respectively.

### SUMMARY

In general, the present disclosure relates to a metered dose inhaler that includes a container and a metering valve connected to the container. The metering valve can include a diaphragm that includes an opening through which a first portion of a metering valve stem is disposed to form a dynamic seal with the diaphragm. The metering valve can further include a tank seal that includes an opening through which a second portion of the metering valve stem is disposed to form a dynamic seal with the tank seal. In one or more embodiments, at least one of the diaphragm or tank seal includes a polytetrafluoroethylene (PTFE) material. The metered dose inhaler can also include a gasket disposed between a ferrule of the metering valve and the container. In one or more embodiments, the gasket can include a PTFE material.

In one aspect, the present disclosure provides a metered dose inhaler that includes a container having a reservoir that contains a formulation including a propellant and at least one active pharmaceutical ingredient, where the propellant includes at least one of hydrofluoroalkane (HFA) or hydrofluoroolefin (HFO); and a metering valve connected to the container. The metering valve includes a valve housing having a first end and a second end, where the valve housing defines a metering chamber between the first end and the second end of the housing, and where the metering chamber is adapted to receive formulation from the reservoir. The metering valve further includes a metering valve stem that extends through the first end of the valve housing, metering chamber, and second end of the valve housing; a diaphragm disposed at least partially within the first end of the valve housing and including an opening, where a first portion of the metering valve stem is disposed through the diaphragm opening and forms a dynamic seal with the diaphragm; and a tank seal disposed at least partially within the second end of the valve housing and including an opening, where a second portion of the metering valve stem is disposed through the tank seal opening and forms a dynamic seal with the tank seal. At least one of the diaphragm or tank seal consists essentially of polytetrafluoroethylene (PTFE).

In another aspect, the present disclosure provides a metered dose inhaler that includes a container including a reservoir that contains a formulation that includes a propellant and at least one active pharmaceutical ingredient, where the propellant includes at least one of HFA or HFO; and a metering valve connected to the container. The metering valve includes a valve housing having a first end and a second end disposed in the reservoir, where the valve housing defines a metering chamber between the first end and the second end of the valve housing, and where the metering chamber is adapted to receive the formulation from the reservoir. The metering valve further includes a metering valve stem that extends through the first end of the valve housing, metering chamber, and second end of the valve housing; a diaphragm disposed at least partially within the first end of the valve housing and including an opening, where a first portion of the metering valve stem is disposed through the diaphragm opening and forms a dynamic seal with the diaphragm; and a tank seal disposed at least partially within the second end of the valve housing and including an opening, where a second portion of the metering valve stem is disposed through the tank seal opening and forms a dynamic seal with the tank seal. At least one of the diaphragm or tank seal consists essentially of polytetrafluoroethylene (PTFE). The metering valve further includes a primed position where the formulation can flow between the reservoir and the metering chamber, and where the diaphragm is adapted to seal the metering chamber from an outside atmosphere. The metering valve further includes an actuated position where the second portion of the metering valve stem seals against the tank seal and the second end of the valve housing to seal the metering chamber from the reservoir, thereby defining a metered volume of formulation within the metering chamber, and where the metering valve stem includes an exit chamber allowing the formulation to flow between the metering chamber and the outside atmosphere.

In another aspect, the present disclosure provides a method that includes disposing a formulation that includes a propellant and at least one active pharmaceutical ingredient within a reservoir of a container, where the propellant includes at least one of hydrofluoroalkane (HFA) or hydrofluoroolefin (HFO); connecting a metering valve to the container; and sealing at least one interface in the valve or between the container and the valve with a PTFE seal.

All headings provided herein are for the convenience of the reader and should not be used to limit the meaning of any text that follows the heading, unless so specified.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements. The term “consisting of” means “including,” and is limited to whatever follows the phrase “consisting of.” Thus, the phrase “consisting of” indicates that the listed elements are required or mandatory and that no other elements may be present. The term “consisting essentially of” means including any elements listed after the phrase and is limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

In this application, terms such as “a,” “an,” and “the” are not intended to refer to only a singular entity but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

These and other aspects of the present disclosure will be apparent from the detailed description below. In no event, however, should the above summaries be construed as limitations on the claimed subject matter, which subject matter is defined solely by the attached claims, as may be amended during prosecution.

### BRIEF DESCRIPTION OF THE DRAWINGS

Throughout the specification, reference is made to the appended drawings, where like reference numerals designate like elements, and wherein:

FIG. 1 is a schematic side view of one embodiment of an inhaler.

FIG. 2 is a schematic side cross-section view of the inhaler of FIG. 1.

FIG. 3 is a schematic side cross-section view of a metering valve of the inhaler of FIG. 1 with the metering valve in a primed position.

FIG. 4 is a schematic side cross-section view of the metering valve of FIG. 3 in an actuated position.

FIG. 5 is a flowchart of one embodiment of a method of forming the inhaler of FIG. 1.

### DETAILED DESCRIPTION

In general, the present disclosure relates to a metered dose inhaler that includes a container and a metering valve connected to the container. The metering valve can include a diaphragm that includes an opening through which a first portion of a metering valve stem is disposed to form a dynamic seal with the diaphragm. The metering valve can further include a tank seal that includes an opening through which a second portion of the metering valve stem is disposed to form a dynamic seal with the tank seal. In one or more embodiments, at least one of the diaphragm or tank seal includes a polytetrafluoroethylene (PTFE) material. The metered dose inhaler can also include a gasket disposed between a ferrule of the metering valve and the container. In one or more embodiments, the gasket can include a PTFE material.

One or more embodiments of metered dose inhalers described herein can provide various advantages over currently-available inhalers. For example, one or more of these embodiments can include one or more seals that include a PTFE material. In typical inhalers, one or more seals instead include elastomeric materials. In one or more embodiments, PTFE seals can have lower levels of leachable materials when compared to elastomeric seals. Further undesirable materials may not need to be extracted from these seals prior to use. PTFE seals are also less likely than standard seals to expand when exposed to propellants. Such PTFE seals can also lower friction between a seal and the valve stem and therefore improved valve function (which can reduce or eliminate the need for silicone or other lubrication), higher resistance to pressure and temperature, and lower permeability when compared to elastomeric seals. PTFE seals can also be used with a wider range of formulation types than can elastomeric seals.

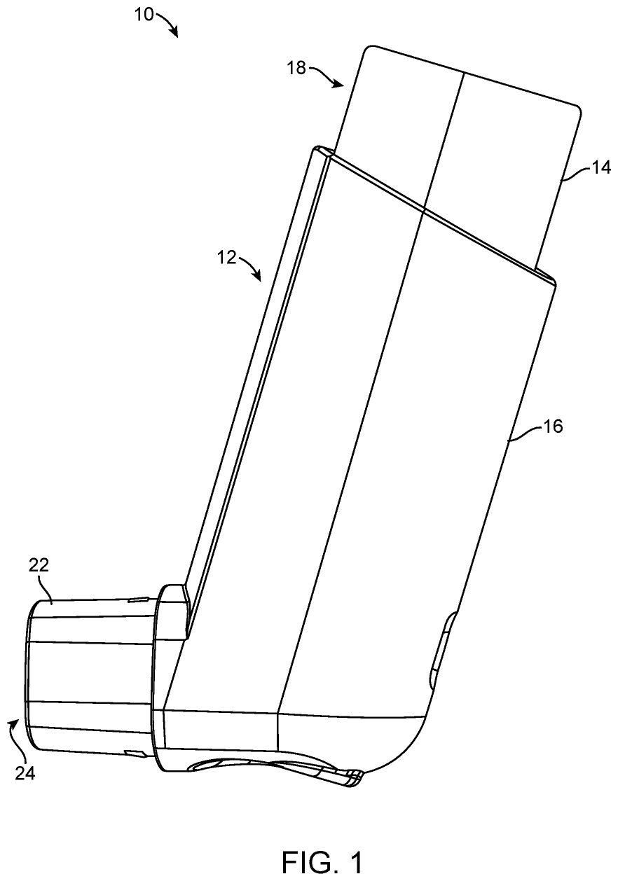

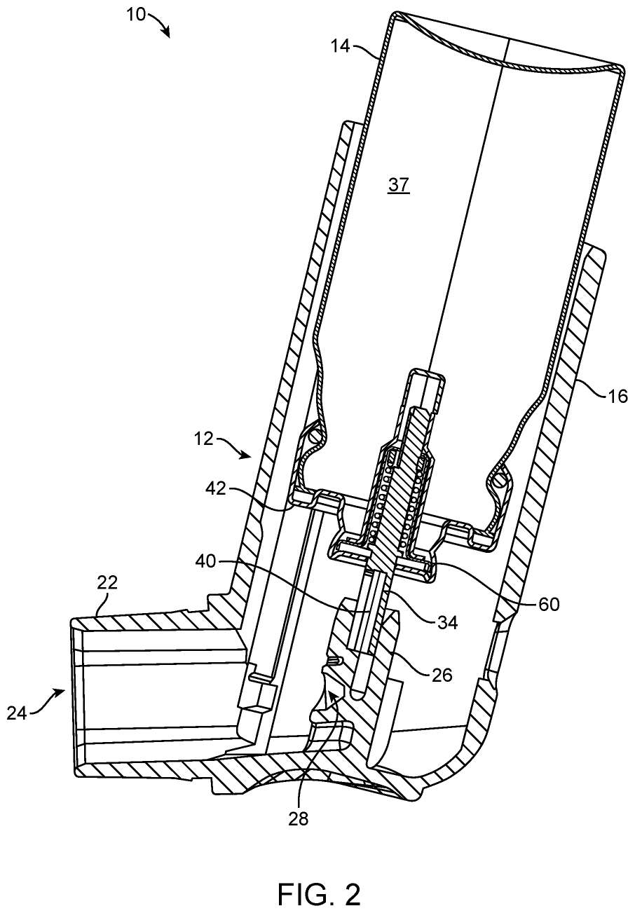

FIG. 1 is a schematic side view of one embodiment of a metered dose inhaler 10. Such inhaler 10 includes an actuator 12 and a container or canister 14. Actuator 12 has a generally elongate actuator housing 16 that houses the container 14. Such container 14 can be inserted into a container opening 18 at the top of the actuator 12. As shown in FIG. 2, which is a schematic side cross-section view of the inhaler 10, a reservoir 37 is disposed within the container 14. The reservoir 37 contains a formulation that includes a propellant and at least one active pharmaceutical ingredient (API) for delivery to a user via the actuator 12 and a mouthpiece 22 of the housing 16 as will be described in further detail herein. In one or more embodiments, the mouthpiece 22 can be replaced by a nosepiece (not depicted) to enable nasal delivery.

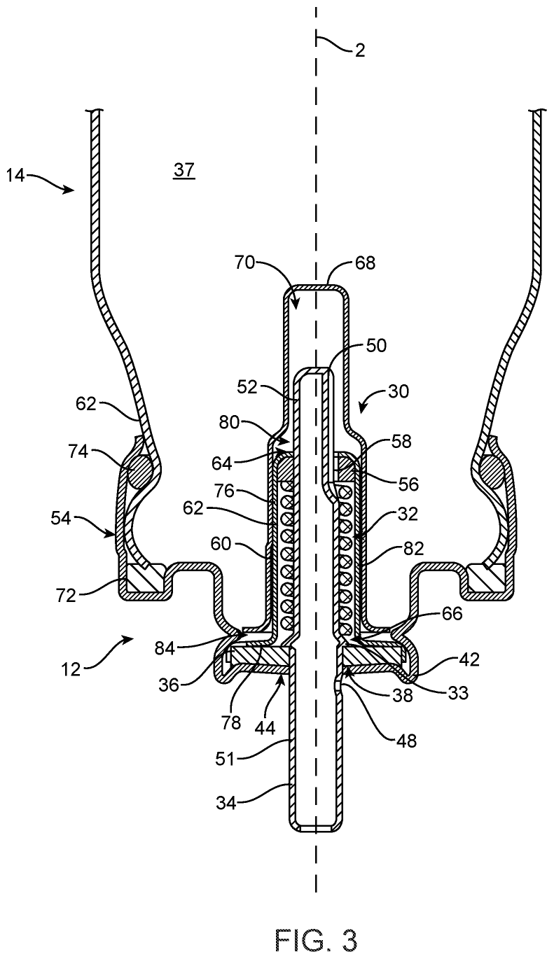

The inhaler 10 also includes a metering valve 30. As shown in FIG. 3, the metering valve 30 includes a valve housing 60 having a first end 78 and a second end 80. The valve housing 60 defines a metering chamber 32 between the first end 78 and the second end 80 of the housing. The metering chamber 32 is adapted to receive formulation from the reservoir 37. The metering valve 30 also includes a metering valve stem 34 that extends through the first end 78, metering chamber 32, and second end 80 of the valve housing 60. Further, the metering valve 30 includes a diaphragm 36 disposed at least partially within the first end 78 of the valve housing 60. The diaphragm 36 includes an opening 38. A first portion 51 of the metering valve stem 34 is disposed through the diaphragm opening 38 and forms a dynamic seal with the diaphragm. The metering valve 30 can also include a tank seal 56 disposed at least partially within the second end 80 of the valve housing 60. The tank seal 56 can include an opening 58. A second portion 52 of the metering valve stem 34 is disposed through the tank seal opening 58 and forms a dynamic seal with the tank seal 56. In one or more embodiments, at least one of the diaphragm 36 or the tank seal 56 can include a polytetrafluoroethene (PTFE) material (i.e., at least one of the diaphragm or tank seal can be a PTFE seal). In one or more embodiments, at least one of the diaphragm or tank seal consists essentially of polytetrafluoroethylene (PTFE).

The inhaler 10 can include any suitable inhaler. In one or more embodiments, the inhaler 10 can include a pressurized metered dose inhaler (pMDI). As shown in FIG. 1, the inhaler 10 includes the container 14 that holds the formulation and the metering valve 30 connected to the container. The container 14 can include any suitable material and take any suitable shape. In one or more embodiments, one or more layers of material can be disposed over at least a portion of the container 14. Such one or more layers can include any suitable material, e.g., PTFE.

The container 14 is disposed within the housing 16 of the actuator 12 through the container opening 18. In one or more embodiments, at least a portion of the container 14 can extend beyond the container opening 18 as shown in FIG. 1. The housing 16 includes a tubular sleeve portion 20 having the container opening 18 that is adapted to receive the container 14, and a portion that defines the mouthpiece 22. Although referred to herein as a mouthpiece 22, such mouthpiece can instead be adapted to be nosepieces of nasal inhalers and that the present disclosure can equally apply to nasal inhalers even where not specifically mentioned herein. The mouthpiece 22 defines an inspiration outlet orifice (e.g., air outlet) 24. In one or more embodiments, the container 14 can include an aspiration orifice or air inlet (not shown) disposed in any suitable portion of the container. The actuator housing 16 can be adapted to enclose at least a portion of the metering valve 30 and the container 14. In one or more embodiments, the actuator housing 16 is adapted to enclosure the metering valve 30 as shown in FIG. 2.

The reservoir 37 disposed within the container 14 can take any suitable shape and have any suitable dimensions. Further, the reservoir 37 can have any suitable internal pressure of formulation. In one or more embodiments, the pressure within the reservoir is no greater than 20 bar. In one or more embodiments, the pressure within the reservoir is in a range of 1-20 bar, 1-15 bar, 1-10 bar, or 1-5 bar.

The inhaler 10 can include any suitable metering valve 30. As shown in FIG. 3, the metering valve 30 includes the valve stem 34 that generally defines a longitudinal axis 2, and the valve housing 60 (i.e., metering tank), where the first portion 51 of the valve stem extends through a central aperture 66 of the first end 78 of the valve housing. In one or more embodiments, the reservoir 37 and metering valve stem 34 are aligned along the central longitudinal axis 2. The valve housing 60 includes the first end 78, the second end 80 disposed in the reservoir 37, and walls 82 that extend between the first end and the second end. The valve housing 60 defines the metering chamber 32 between the first end 78 and the second end 80 of the housing.

The valve housing 60 can take any suitable shape and having any suitable dimensions. Further, the metering chamber 32 defined by the valve housing can also take any suitable shape and have any suitable dimensions. In one or more embodiments, the metering chamber 32 has a volume that may be between 5 microliters (μL or mcl) and 200 microliters, between 25 microliters and 200 microliters, between 25 microliters and 150 microliters, between 25 microliters and 100 microliters, between 50 microliters and 100 microliters, between 25 microliters and 65 microliters, or between 50 microliters and 65 microliters.

Extending through the first end 78, the metering chamber 32, and the second end 80 of the valve housing 60 is the metering valve stem 34. Such stem 34 includes the first portion 51 that extends through the first end 78 of the valve housing 60 and is exposed to atmosphere, and the second portion 52 that extends through the second end 80 of the valve housing. The first portion 51 of the valve stem 34 extends outwardly and is in slidable, sealing engagement with the diaphragm 36, while the second portion 52 of the valve stem extends inwardly and is in slidable, sealing engagement with the tank seal 56.

The metering valve 30 is connected to the container 14 using any suitable technique. As shown in FIG. 3, the metering valve 30 is connected to the container 14 by a ferrule 42 that can be connected to the container using any suitable technique. In one or more embodiments, the ferrule 42 is mechanically connected to the container 14, e.g., by crimping the ferrule to the container. The ferrule 42 includes a mounting 54 that is connected to the container 14 to define the reservoir 37. A gasket 72 can be disposed between the ferrule 42 and an end 15 of the container 14. The gasket 72 can be adapted to seal the reservoir 37 and the ferrule 42. Further, an O-ring 74 can be disposed between the ferrule 42 and a side surface 62 of the container 14 to further seal the reservoir 37 from the outside atmosphere.

An aperture 44 is disposed through the ferrule 42, and the opening 38 of the diaphragm 36 is aligned with the aperture of the ferrule. The first portion 51 of the valve stem 34 extends through the opening 38 of the diaphragm 36 and is slidably engaged with the opening. The diaphragm 36, therefore, forms the dynamic seal with valve stem 34. Further, the diaphragm 36 forms a dynamic seal between the metering chamber 32 and the outside atmosphere. As used herein, the phrase “dynamic seal” means that a seal is maintained with a moving part that substantially prevents a liquid or gas from passing through an opening of the seal as the moving part is articulated through the seal. In one or more embodiments, the diaphragm 36 can be sealed to the ferrule 42 using any suitable technique. The diaphragm 36 can include a spring (not shown) disposed within the diaphragm or in contact with an exterior surface of the diaphragm that is adapted to maintain the seal in contact with the ferrule 42. In one or more embodiments, the diaphragm 36 does not include a spring.

The second portion 52 of the valve stem 34 is disposed through the opening 58 of the tank seal 56 and is in slidable engagement with such opening. The second portion 52 of the valve stem 34 forms a dynamic seal with the tank seal 56. In one or more embodiments, a dynamic seal can also be formed by the tank seal 56 between the metering chamber 32 and a second valve housing 68. The second portion 52 of the valve stem 34 passes through an inlet aperture 64 at the second end 80 of the valve housing 60.

The valve stem 34 is in slidable engagement with the opening 38 of the diaphragm 36. A compression spring 46 is adapted to hold the valve stem 34 in a primed position as illustrated in FIG. 3. The compression spring 46 can be disposed within the valve housing 60 between the tank seal 56 and a flange 33 disposed on the valve stem 34. The valve stem 34 further includes an orifice 48 that is adapted to fluidly communicate with an exit chamber 40 that is disposed in the valve stem, and a channel 50 that is disposed in first portion 51 of the valve stem.

In one or more embodiments, the metering valve 30 can include a second valve housing 68 (i.e., retaining cup) that defines a bottle emptier. When such second valve housing 68 is present, formulation in the reservoir 37 can pass through a gap 84 between the valve housing 60 and the second valve housing 68, through an annular gap 76 into a retention chamber 70 (i.e., retention chamber), and then through the channel 50 disposed in the valve stem 34 and into the metering chamber 32. The second valve housing 68 can be a connected to the ferrule 42 using any suitable technique. The annular gap 76 of the retention chamber 70 can be present when the inhaler 10 is adapted to be used with a suspension aerosol formulation. In one or more embodiments, when the inhaler 10 is adapted to be used with a solution aerosol formulation, the second valve housing 68 may be optional.

As shown in FIG. 2, the valve stem 34 protrudes from the valve housing 60 and the ferrule 42 and is connected to a stem socket 26 that is disposed within the housing 16. A spray orifice 28 is disposed in the stem socket 26 and provides fluid communication between the valve stem 34 and the inspiration orifice 24. A user can place the mouthpiece 22 into a body cavity (e.g., mouth) and inhale through it while pressing downwards on the container 14. This pressing force moves the container 14 toward the stem socket 26 relative to the valve stem 34. The relative movement isolates a metered dose of the pressurized formulation from the bulk formulation disposed in the reservoir 37 of the container 14 and then discharges it via an exit chamber 40 disposed within the stem 34. The discharged dose passes along a fluid passageway through the stem socket 26 and the spray orifice 28 and is directed into the user's body cavity (e.g., at least one of an oral cavity or nasal cavity) and into the user's respiratory passages.

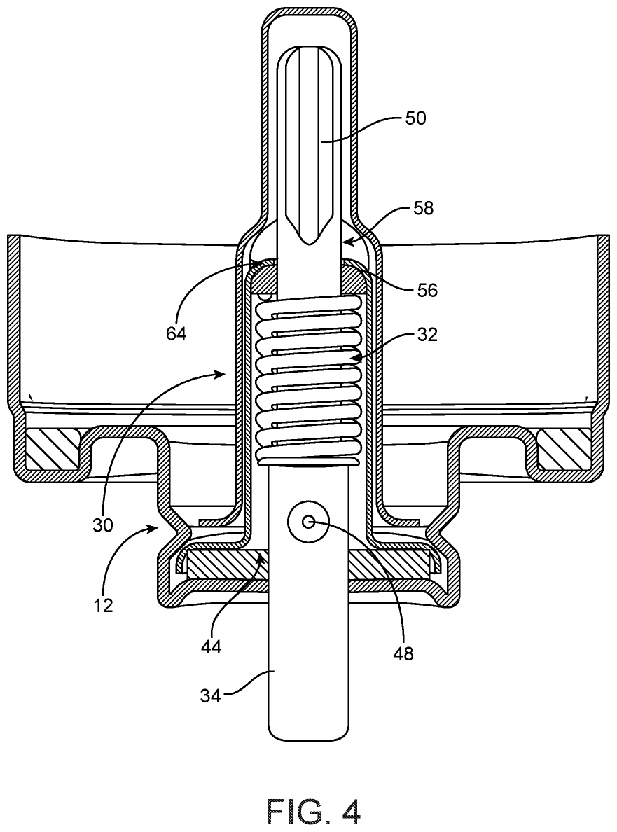

Operation of the inhaler 10 is illustrated in FIGS. 3-4. In FIG. 3, the inhaler 10 is adapted such that the valve 30 is in the primed position, where formulation can flow between the reservoir 37 and the metering chamber 32, and where the diaphragm 36 is adapted to seal the metering chamber from the outside atmosphere. The annular gap 76 allows open communication between the retention chamber 70 and the reservoir 37, thus allowing the formulation in the reservoir to enter the retention chamber 70. Channel 50 in the metering stem 34 allows open communication between the retention chamber 70 and the metering chamber 32 such that a portion of the formulation enters the metering chamber 32 through the inlet aperture 64. The diaphragm 36 seals the central aperture 66 of the valve housing 60.

FIG. 4 is a schematic cross-section view of a portion of the inhaler 10 in an actuated position, where formulation can flow between the metering chamber 32 and the outside atmosphere. In the actuated position, the second portion 52 of the metering valve stem seals against the tank seal 56 and the second end 80 of the valve housing 60 to seal the metering chamber 32 from the reservoir 37, thereby defining a metered volume of formulation within the metering chamber. As the valve stem 34 is depressed, channel 50 is moved relative to the tank seal 56 such that the inlet aperture 64 and the tank seal opening 58 are substantially sealed, thus isolating a metered dose of formulation within the metering chamber 32. Further depression of the valve stem 34 can cause the orifice 48 to pass through the aperture 44 of the ferrule 42 and into the metering chamber 32, where the metered dose is exposed to ambient pressure. Rapid vaporization of the propellant of the formulation can cause the metered dose to be forced through the orifice 48 and into and through the exit chamber 40. In other words, the exit chamber 40 allows the formulation to flow between the metering chamber 32 and the outside atmosphere. It should be understood that other modes of actuation, such as breath-actuation, may be used as well and would operate as described with the exception that the force to depress the container would be provided by the device, for instance by a spring or a motor-driven screw, in response to a triggering event, such as patient inhalation.

In general, the inhaler 10 can include one or more seals or gaskets (e.g., diaphragm 36) that are adapted to seal at least one interface between elements or components of the inhaler or at least one interface between elements or components of the inhaler and the outside atmosphere. For example, one or more seals can seal at least one interface in the metering valve 30 or between the container 14 and the valve. In one or more embodiments, the at least one interface can be between the end 15 of the container 14 and the ferrule 42. In one or more embodiments, the at least one interface is between the metering valve stem 34 and the outside atmosphere. Further, in one or more embodiments, the at least one interface is between the metering chamber 32 and the reservoir 37.

In one or more embodiments, the inhaler 10 can include at least three distinct seals, i.e., the diaphragm 36, the tank seal 56, and the gasket 72. In one or more embodiments, the inhaler 10 can also include the O-ring 74. The seals can include any suitable material. In one or more embodiments, each of the seals can include the same materials. In one or more embodiments, one or more of the seals can include a material that is different from the material utilized for one or more additional seals. In one or more embodiments, at least one of the seals can include a PTFE material. For example, at least one of the diaphragm 36, tank seal 56, gasket 72, or O-ring 74 can include PTFE. In one or more embodiments, at least one of the diaphragm 36, tank seal 56, gasket 72, or O-ring 74 can consist essentially of PTFE.

Exemplary materials used to form the seals, can include, but are not limited to, PTFE, ethylene tetrafluoroethylene (ETFE), fluorinated ethylene propylene (FEP), polyethylene, particularly high-density polyethylene or linear low-density polyethylene, polyamide, such as nylon, and polypropylene. Further, each of the seals can have any suitable hardness, e.g., a hardness of about 45 to 80, 50 to 70, 50 to 60, or 55 to 60 Shore D as measured by ASTM D2240-1 5 in a standard atmosphere of 23° C. and 50% relative humidity.

As mentioned herein, the inhaler 10 can include any suitable metering valve 30. Further, the metering valve 30 can exhibit any suitable characteristics. For example, the metering valve 30 can include any suitable start of life leak rate. As used herein, the phrase “start of life leak rate” means the weight of formulation, or specific formulation components, exiting the canister per unit of time when the valve is in its primed state and none of the labelled claimed doses have been dispensed. In one or more embodiments, the start of life leak rate is no greater than 650 mg/yr. In one or more embodiments, the start of life leak rate is no greater than 300 mg/yr.

The inhaler 10 can include any suitable formulation disposed within reservoir 37, e.g., one or more of the formulations described in PCT Patent Application Nos. PCT/US2022/042956, filed 8 Sep. 2022, and entitled METERED DOSE INHALERS AND SOLUTION COMPOSITIONS; PCT/US2022/042958, filed 8 Sep. 2022, and entitled METERED DOSE INHALERS AND SUSPENSION COMPOSITIONS; and PCT/US2022/042959, filed 8 Sep. 2022, and entitled PROPELLANTS FOR ANTICHOLINERGIC AGENTS IN PRESSURIZED METERED DOSE INHALERS. In one or more embodiments, the formulation includes at least one propellant and at least one API. The formulation can include any suitable propellant. In one or more embodiments, the propellant can include at least one of a hydrofluoroalkane (HFA) or hydrofluoroolefin (HFO). The primary propellant of compositions (i.e., formulations) according to the disclosure is HFO-1234ze(E), also known as trans-1,1,1,3-tetrafluoropropene, trans-1,3,3,3-tetrafluoropropene, or trans-1,3,3,3-tetrafluoroprop-1-ene. The chemical structure of trans and cis isomers of HFO-1234ze are very different. As a result, these isomers have very different physical and thermodynamic properties. The significantly lower boiling point and higher vapor pressure of the trans (E) isomer relative to that of the cis (Z) isomer, at ambient conditions, makes the trans isomer a far more thermodynamically suitable propellant for achieving efficient pMDI atomization.

In one or more embodiments, the amount of HFO-1234ze(E) by weight in the composition is greater than 70%, at least 80%, greater than 80%, at least 85%, greater than 85%, at least 90%, or greater than 90%. In some embodiments, the amount of HFO-1234ze(E) by weight is between 80% and 99%, between 80% and 98%, between 80% and 95%, or between 85% and 90%. In one or more embodiments, HFO-1234ze(E) is essentially the sole propellant in the composition. That is, the pharmaceutical product performance parameters, such as emitted dose and emitted particle size distribution, are not significantly different than if HFO-1234ze(E) were the sole propellant in the composition. In some embodiments, the amount of HFO-1234ze(E) by weight of the total propellant in the composition is greater than 95%, greater than 98%, greater than 99%, greater than 99.5%, and greater than 99.8%.

The propellant HFO-1234ze(E) is very different from an alternative low GWP propellant HFA-152a. These two propellants have different physical, chemical, and thermodynamic properties such as boiling point, vapor pressure, water solubility, liquid density, surface tension, etc. The differences in these properties make replacing one propellant with another without significantly compromising or altering pMDI product performance difficult to achieve. For example, the thermodynamic differences in propellant boiling point and vapor pressure can significantly affect pMDI aerosolization efficiency and give rise to differences in primary and secondary atomization mechanisms. Differences in dipole moment and polarity between the propellants can affect the solubility of drugs and excipients in the formulation. Differences in hygroscopicity between the propellants can affect moisture uptake, which could be problematic for solution formulations, particularly if physical stability due to moisture uptake or chemical degradation in which water is involved is likely. Chemical interactions of the different propellants with drug and excipients may also be significantly different, which could affect the long-term chemical stability of the product over the intended shelf life. The two propellants interact chemically and physically with valve plastics and elastomeric components, which could give rise to differences in the types and amounts of extractables and leachables, as well as affecting mechanical valve function. The thermodynamic properties of the propellants can give rise to different droplet particle sizes due to different evaporation rates and can also result in differences in spray characteristics such as spray force, temperature, and spray duration. Historically, the transition from CFC to HFA propellants has required significant efforts to develop new approaches to reformulate and develop capable hardware to achieve appropriate pMDI product performance. That is, it was not possible to simply directly substitute one propellant for another. Changing between propellant HFA-152a to HFO-1234ze(E) in a pMDI is equally challenging due to many of the factors highlighted above.

In one or more embodiments, other propellants, such as hydrofluoroalkanes, including HFA-134a, HFA-227 (1,1,1,2,3,3,3-heptafluoropropane), or HFA-152a, may be included as a minor component. Still other propellants that may be included as a minor component include other hydrofluoroolefins, including HFO-1234yf (2,3,3,3-tetrafluoropropene) and HFO-1234ze (Z) (i.e., cis-HFO-1234ze). Thus, in one or more embodiments, the differences between HFA-152a and HFO-1234ze(E) discussed herein can be utilized to advantage by using a minor amount of HFA-152a. Amounts of such secondary propellants can include 0.1% to 20%, 0.1% to 5%, or 0.1% to 0.5%, by weight, of the total composition (i.e., total formulation).

In one or more embodiments, a cosolvent is included. One particularly useful cosolvent is ethanol. In one or more embodiments, ethanol is used as a cosolvent in solution formulations, i.e., where the API is dissolved in the formulation. In one aspect, the ethanol may aid in dissolving the API whereas the API may not be soluble in the formulation in the absence of ethanol. When used in solution formulations, ethanol may be in amounts on a weight percent basis of the total formulation of at least 0.5%, at least 1%, at least 2%, at least 5%, at least 10%, at least 15%. When used in solution formulations, ethanol may be in amounts on a weight percent basis of the total formulation of up to 20% or up to 15%.

Further, the formulation can include any suitable API. The API may be a drug, vaccine, DNA fragment, hormone, other treatment, or a combination of any two or more APIs. In one or more embodiments, the formulations may include at least two (in certain embodiments, two or three, and in certain embodiments, two) APIs in solution.

The API may be provided in any form suitable for formulation as a solution. In one or more embodiments, the API may be provided as a solid, such as a powder or a micronized powder, or as a liquid, such as a stock solution. Any suitable form of API compatible with preparation of a solution may be used for the formulations of the present disclosure.

Exemplary APIs can include those for the treatment of respiratory disorders, e.g., a bronchodilator, such as a short- or long-acting beta agonist, an anti-inflammatory (e.g., a corticosteroid), an anti-allergic, an anti-asthmatic, an antihistamine, a TYK inhibitor, or an anticholinergic agent. Exemplary APIs can include terbutaline, ipratropium, oxitropium, tiotropium, beclomethasone, flunisolide, ciclesonide, cromolyn sodium, nedocromil sodium, ketotifen, azelastine, ergotamine, cyclosporine, aclidinium, umeclidinium, glycopyrronium (i.e., glycopyrrolate), salmeterol, formoterol, procaterol, indacaterol, carmoterol, milveterol, olodaterol, vilanterol, abediterol, omalizumab, zileuton, insulin, pentamidine, calcitonin, leuprolide, alpha-I-antitrypsin, interferon, triamcinolone, nintedanib, a pharmaceutically acceptable salt or ester of any of the listed drugs, or a mixture of any of the listed drugs, their pharmaceutically acceptable salts or their pharmaceutically acceptable esters. For beclomethasone, an exemplary ester is propionate.

In all embodiments, the API(s) are dissolved in the formulation (i.e., as a solution). In the event that a combination of two or more APIs are used, all of the APIs are in solution.

In one embodiment, the formulation has beclomethasone or a pharmaceutically acceptable salt or ester thereof as the sole API, more particularly beclomethasone dipropionate.

In one embodiment, the formulation has formoterol or a pharmaceutically acceptable salt or ester thereof as the sole API, more particularly formoterol fumarate.

In one embodiment, the formulation includes tiotropium or a pharmaceutically acceptable salt or ester thereof as the sole API, more particularly tiotropium bromide.

In one embodiment, the formulation includes beclomethasone and formoterol or pharmaceutically acceptable salts or esters thereof, more particularly beclomethasone dipropionate and formoterol fumarate, and more particularly where both active ingredients are dissolved in the formulation.

The amount of API may be determined by the required dose per actuation and the pMDI metering valve size, that is, the size of the metering chamber 32. The metering chamber 32 can include any suitable volume as is further described herein. The concentration of each API is typically from 0.0008% to 3.4% by weight, or 0.01% to 1.0% by weight, sometimes from 0.05% to 0.5% by weight, and as such, the medicament makes up a relatively small percentage of the total composition.

In certain embodiments, typical formulations of the present disclosure include the API in an amount of at least 0.001 milligram per actuation (mg/actuation), or at least 0.001 mg/actuation. In one or more embodiments, typical formulations of the present disclosure include the API in an amount of less than 0.5 mg/actuation.

In one or more embodiments, typical formulations of the present disclosure include the API in an amount of at least 1 μg/actuation, at least 10 μg/actuation, at least 50 μg/actuation, at least 100 μg/actuation, at least 150 μg/actuation, at least 200 μg/actuation, at least 300 μg/actuation, or at least 400 μg/actuation. In embodiments, typical formulations of the present disclosure include the API in an amount of less than 500 μg/actuation, at most 400 μg/actuation, at most 300 μg/actuation or at most 200 μg/actuation. In some preferred embodiments, formulations of the present disclosure include the API in an amount of 80 μg/actuation to 120 μg/actuation.

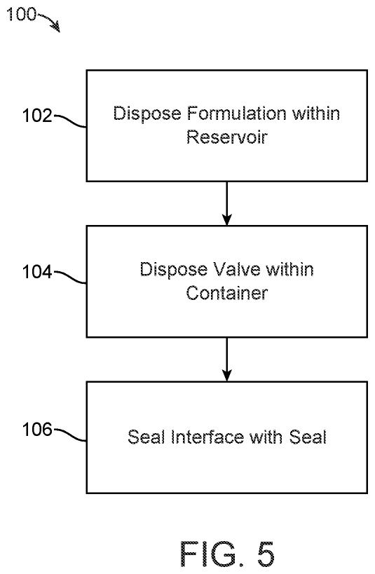

Any suitable technique can be utilized to form the inhaler. For example, FIG. 5 is a flowchart of one method 100 of forming the inhaler 10. Although described regarding the inhaler 10 of FIGS. 1-4, the method 100 can be utilized to form any suitable inhaler. At 102, the formulation is disposed within the reservoir 37 of the container 14 using any suitable technique.

The metering valve 30 can be connected to the container 14 at 104 using any suitable technique. The formulation can be disposed within the reservoir prior to connecting the metering valve 30 to the container 14 or after connecting the metering valve to the container. In one or more embodiments, the valve stem 34 of the metering valve 30 can be disposed through the aperture 44 of the ferrule 42, and the ferrule can be connected to the end 15 of the container 14 such that the metering valve is disposed at least partially within the container 14. In one or more embodiments, the diaphragm 36 can be disposed within the ferrule 42 such that the opening 38 of the diaphragm is aligned with the aperture 44 of the ferrule and the valve stem 34 is disposed through the opening of the diaphragm and the aperture of the ferrule.

At 106, at least one interface in the valve 30 or between the container 14 and the valve can be sealed with a seal using any suitable techniques. In one or more embodiments, the seal can be a PTFE seal. Any suitable interface can be sealed utilizing a seal. In one or more embodiments, the at least one interface includes an interface between the end 15 of the container 14 and the ferrule 42 that can be sealed with the gasket 72 (e.g., a PTFE gasket). Further, in one or more embodiments, the at least one interface includes an interface between the metering valve stem 34 and the diaphragm 36 to form a dynamic seal between the metering valve stem and the diaphragm. Further, in one or more embodiments, the at least one interface includes an interface between the metering chamber 32 of the metering valve 30 and the reservoir 37 with the tank seal 56 to form a dynamic seal between the metering valve stem and the tank seal. In such embodiments, a portion (e.g., second portion 52) of the valve stem 34 is disposed through the opening 58 of the tank seal 56.

### EXAMPLES

An ethanolic solution/suspension was prepared using the drug of interest at the required concentration. A volume of the solution/suspension was pipetted into a 16 ml canister that was coated inside with fluorinated ethylene propylene. The required amount of propellant was cold transferred into the canister. A ferrule of a metering valve was crimped onto the 16 ml canister. The metering valve was a modified Kindeva™ Spraymiser™ valve core assembly that included a valve stem, spring, outer seal diaphragm, and inner seal tank seal, fitted into the canister ferrule with a metering tank and held in place with a valve retainer. The metering valve further included a PTFE diaphragm made from a 1 mm thick PTFE sheet (Material Q400 (ET) A Actionplas, UK).

After assembly, the canisters were assessed to determine whether they were leaking by looking for weight decreases. They were then stored with the valve oriented downward. After 24 hours, the canisters were function tested by actuating the valve three times to assess that the valves were functioning correctly. After an additional 24 hours, leak testing of the canisters commenced for a period of up to seven days. Following this seven day period, the canisters were further tested for through life (Examples 1-2 and 5-6) and shot weight (Examples 3-4). After completion of this testing, the canisters were once again assessed for leak rate for a seven day period.

### Leakage Testing

After 24 hours post function testing, each canister was weighed on five decimal place balance to determine an initial weight. They were then stored valve up for seven days at ambient and re-weighed on balance.

### Through Life Shot Weight Testing for Examples 3-4

Each canister was placed in a Mark 6 actuator with a 0.3 mm exit orifice diameter (available from Kindeva Drug Delivery), shaken for five seconds with a rolling motion, and actuated once every five seconds for a total of four actuations. The canisters were removed from the actuator and weighed. The canisters were then placed in the actuator, shaken for five seconds with a rolling motion, actuated, and weighed. This was repeated until each canister had been actuated and weight 120 times. The weight of the inhaler (canister plus actuator) was determined before and after each shot, and the shot weight was determined from the difference. The result is reported as the number of shots for the shot weight to reach approximate steady state, the number of shots produced at steady state, and the average and standard deviation of the shot weights at steady state.

Through Life Delivered Dose Testing for Examples 1-2 and 5-6

### Examples 1-2

Through life delivered dose and through life shot weight were assessed using a Mark 6 actuator with a 0.3 mm exit orifice diameter (Kindeva Drug Delivery) at start of life (shots 1 to 3) and middle of life (shots 99 to 102), shaking the inhaler 5 times prior to into unit spray collection apparatus (USCA) fitted with a 25 mm filter (e.g., Whatman Grade 934-AH) at a flow rate of 28.3±0.5 L/min. The sample was recovered using 20 ml of diluent (75:25 Methanol:Water v/v), filtered through a 0.45 μm PVDF syringe filter, and analyzed by reversed phase high performance liquid chromatography using UV detection. The weight of each shot was determined gravimetrically.

### Examples 5-6

Through life delivered dose and through life shot weight was assessed using a Mark 6 actuator with a 0.4 mm exit orifice diameter (Kindeva Drug Delivery) at start of life (shots 1 to 3), middle of life (shots 59 to 62), and end of life (shots 118 to 120), shaking the inhaler 40 times prior to actuating into unit spray collection apparatus (USCA) fitted with a 25 mm filter (e.g., Whatman Grade 934-AH) at a flow rate of 28.3±0.5 L/min. The sample was recovered using 20 ml of diluent (75:25 Methanol:Water v/v), filtered through a 0.45 μm PVDF syringe filter, and analyzed by reversed phase high performance liquid chromatography using UV detection. The weight of each shot was determined gravimetrically.

### Example 1

A solution was prepared of 1.5873 mg/ml beclomethasone dipropionate (Teva) in HFO-1234ze with 8% by weight of ethanol. The solution was added to three refillable 16 mL canisters. The canisters were prepared and tested as described above.

### Example 2

A solution was prepared of 1.5873 mg/ml beclomethasone dipropionate (Teva) in HFA-152a with 8% by weight of ethanol. The solution was added to three refillable 16 mL canisters. The canisters were prepared and tested as described above.

### Example 3

A placebo solution was prepared of HFO-1234ze and was added to two 16 mL canisters. The canisters were prepared and tested as described above.

### Example 4

A placebo solution was prepared of HFA-152a and was added to two 16 mL canisters. The canisters were prepared and tested as described above.

### Example 5

A suspension was prepared of 1.9126 mg/ml salbutamol sulphate (Teva API, Israel) in HFA-152a with 5% by weight of ethanol. The solution was added to three refillable 16 mL canisters. The canisters were prepared and tested as described above.

### Example 6

A suspension was prepared of 1.9126 mg/ml salbutamol sulphate (Teva API, Israel) in HFO-1234ze with 5% by weight of ethanol. The solution was added to three refillable 16 mL canisters. The canisters were prepared and tested as described above.

The following table includes the results of start of life and end of life leak rates for Examples 1-6. Please note that end of life leak rate data for Examples 1-2 were not acquired.

TABLE 1
LEAK RATE

| | | Leak rate (mg/year) | |
|---|---|---|---|
| Formulation description | Canister # | Start of life | End of life |
| Example 1 | 1 | 40.8 | * |
| | 2 | 38.7 | * |
| | 3 | 25.1 | * |
| Example 2 | 1 | 68.0 | * |
| | 2 | 62.3 | * |
| | 3 | 78.0 | * |
| Example 3 | 1 | 54.4 | 123.8 |
| | 2 | 93.7 | 176.4 |
| Example 4 | 1 | 74.3 | 123.3 |
| | 2 | 69.6 | 117.1 |
| Example 5 | 1 | 134.4 | 103.7 |
| | 2 | 119.0 | 93.9 |
| | 3 | 107.3 | 97.2 |
| Example 6 | 1 | 113.2 | 110.2 |
| | 2 | 113.2 | 115.6 |
| | 3 | 97.2 | 95.5 |

The following table includes data for through life shot weight for Examples 3-4 for 120 actuations.

TABLE 2
THROUGH LIFE SHOT WEIGHT

| | |
|---|---|
| Placebo HFO-1234ze | Placebo HFA-152a |

| | Canister | Canister | Canister | Canister |
|---|---|---|---|---|
| Shot number | 1 (mg) | 2 (mg) | 1 (mg) | 2 (mg) |
| Average | 70.1 | 70.8 | 54.7 | 54.6 |
| Maximum | 70.9 | 71.3 | 57.0 | 55.5 |
| Minimum | 69.5 | 70.2 | 50.3 | 53.7 |
| Range | 1.4 | 1.1 | 6.7 | 1.8 |

The following table includes data for mcg/actuation for Examples 1-2 and 5-6. Please note that no end of life data was collected for Examples 1-2.

TABLE 3
THROUGH LIFE DELIVERED DOSE FOR EXAMPLES 1-2 AND 5-6

| | mcg/actuation | | | | | | | | | |
|---|---|---|---|---|---|---|---|---|---|---|
| Formulation | Start of life | | | Middle of life | | | | End of life | | |
| description | 1 | 2 | 3 | 1 | 2 | 3 | 4 | 1 | 2 | 5 |
| Example 1 | 92.794 | 80.919 | 101.244 | 89.914 | 95.808 | 103.075 | 93.966 | * | * | * |
| | 93.496 | 90.323 | 90.527 | 93.800 | 94.371 | 95.199 | 89.995 | * | * | * |
| | 87.020 | 86.525 | 87.165 | 87.067 | 92.155 | 87.086 | 86.926 | * | * | * |
| Example 2 | 103.397 | 110.950 | 110.157 | 106.434 | 111.531 | 106.845 | 105.524 | * | * | * |
| | 94.882 | 97.750 | 98.037 | 96.963 | 105.290 | 101.543 | 101.181 | * | * | * |
| | 98.137 | 99.877 | 102.359 | 104.207 | 106.861 | 103.349 | 106.262 | * | * | * |
| Example 5 | 79.993 | 45.443 | 76.813 | 83.577 | 84.188 | 87.656 | 89.221 | 87.069 | 87.651 | 86.776 |
| | 89.022 | 91.693 | 89.823 | 89.840 | 89.935 | 89.667 | 84.731 | 84.542 | 93.164 | 89.260 |
| | 91.589 | 86.459 | 68.286 | 91.929 | 87.053 | 90.624 | 94.686 | 85.064 | 88.787 | 95.737 |
| Example 6 | 110.246 | 116.500 | 98.873 | 88.963 | 81.333 | 82.473 | 83.921 | 78.965 | 81.251 | 80.191 |
| | 88.111 | 88.212 | 77.392 | 76.773 | 77.990 | 78.693 | 76.070 | 70.547 | 75.784 | 78.329 |
| | 83.183 | 86.568 | 89.947 | 82.980 | 84.656 | 78.791 | 79.916 | 81.773 | 86.221 | 88.232 |

The following table includes through life shot weight data for Examples 1-2 and 5-6. Please note that no end of life data was collected for Examples 1-2.

TABLE 4
THROUGH LIFE SHOT WEIGHT EXAMPLES 1-2 AND 5-6

| | | Shot weight (mg) | | | | | | | | | |
|---|---|---|---|---|---|---|---|---|---|---|---|
| Formulation | Canister | Start of life | | | Middle of life | | | | End of life | | |
| description | # | 1 | 2 | 3 | 1 | 2 | 3 | 4 | 1 | 2 | 3 |
| Example 1 | 1 | 67.3 | 67.5 | 66.7 | 68.6 | 64.9 | 66.8 | 67.0 | * | * | * |
| | 2 | 68.3 | 67.1 | 66.7 | 69.1 | 66.5 | 66.7 | 67.4 | * | * | * |
| | 3 | 67.3 | 67.2 | 68.5 | 67.1 | 67.0 | 67.8 | 67.7 | * | * | * |
| Example 2 | 1 | 53.5 | 53.9 | 54.2 | 54.7 | 53.1 | 53.7 | 53.8 | * | * | * |
| | 2 | 53.5 | 54.4 | 53.4 | 54.0 | 55.1 | 52.7 | 54.7 | * | * | * |
| | 3 | 53.9 | 54.2 | 53.8 | 54.1 | 54.8 | 53.2 | 54.8 | * | * | * |
| Example 5 | 1 | 68.0 | 68.0 | 68.0 | 69.0 | 69.0 | 69.0 | 69.0 | 69.3 | 69.3 | 69.3 |
| | 2 | 68.2 | 68.2 | 68.2 | 68.3 | 68.3 | 68.3 | 68.3 | 68.8 | 68.8 | 68.8 |
| | 3 | 59.2 | 59.2 | 59.2 | 68.8 | 68.8 | 68.8 | 68.8 | 68.8 | 68.8 | 68.8 |
| Example 6 | 1 | 45.0 | 45.0 | 45.0 | 54.2 | 54.2 | 54.2 | 54.2 | 54.5 | 54.5 | 54.5 |
| | 2 | 42.7 | 42.7 | 42.7 | 54.8 | 54.8 | 54.8 | 54.8 | 54.6 | 54.6 | 54.6 |
| | 3 | 42.9 | 42.9 | 42.9 | 54.6 | 54.6 | 54.6 | 54.6 | 54.5 | 54.5 | 54.5 |

The data demonstrated that the use of PTFE as the sealing material produced a metering valve with a low start of life and end of life leak rate (<175 mg/year), consistent through life shot weights, and satisfactory through life delivered dose for both solution and suspension formulations in HFA-152a and HFO-1234ze (Z).

The invention is defined in the claims; however, below there is provided a non-exhaustive listing of non-limiting examples. Any one or more of the features of these examples may be combined with any one or more features of another example, embodiment, or aspect described herein.

Example Ex1. A metered dose inhaler that includes a container having a reservoir that contains a formulation including a propellant and at least one active pharmaceutical ingredient, where the propellant includes at least one of hydrofluoroalkane (HFA) or hydrofluoroolefin (HFO); and a metering valve connected to the container. The metering valve includes a valve housing having a first end and a second end, where the valve housing defines a metering chamber between the first end and the second end of the housing, and where the metering chamber is adapted to receive formulation from the reservoir. The metering valve further includes a metering valve stem that extends through the first end of the valve housing, metering chamber, and second end of the valve housing; a diaphragm disposed at least partially within the first end of the valve housing and including an opening, where a first portion of the metering valve stem is disposed through the diaphragm opening and forms a dynamic seal with the diaphragm; and a tank seal disposed at least partially within the second end of the valve housing and including an opening, where a second portion of the metering valve stem is disposed through the tank seal opening and forms a dynamic seal with the tank seal. At least one of the diaphragm or tank seal consists essentially of polytetrafluoroethylene (PTFE).

Example Ex2. The inhaler of Ex1, where the diaphragm does not include a spring.

Example Ex3. The inhaler of any one of Ex1 to Ex2, where a pressure within the reservoir is no greater than 20 bar.

Example Ex4. The inhaler of any one of Ex1 to Ex3, where at least one of the diaphragm or tank seal includes a Shore D hardness of 50 to 60.

Example Ex5. The inhaler of any one of Ex1 to Ex4, where the reservoir and metering valve stem are aligned along a central longitudinal axis.

Example Ex6. The inhaler of any one of Ex1 to Ex5, where a volume of the metering chamber is about 25 to 100 microliters.

Example Ex7. The inhaler of any one of Ex1 to Ex6, further including an actuator having an actuator housing adapted to enclose at least a portion of the metering valve and container.

Example Ex8. The inhaler of Ex7, further including a ferrule that is adapted to connect to the metering valve to an end of the container, and a gasket disposed between the ferrule and the first end of the container.

Example Ex9. The inhaler of Ex8, where the gasket consists essentially of PTFE.

Example Ex10. The inhaler of any one of Ex8 to Ex9, further including an O-ring disposed between the ferrule and a side surface of the cannister, where the O-ring consists essentially of PTFE.

Example Ex11. The inhaler of any one of Ex1 to Ex10, where each of the diaphragm and tank seal consists essentially of PTFE.

Example Ex12. The inhaler of any one of Ex1 to Ex11, where the metering valve further includes a second valve housing disposed over the second end of the valve housing.

Example Ex13. The inhaler of any one of Ex1 to Ex12, where the propellant includes HFO-1234ze(E).

Example Ex14. The inhaler of any one of Ex1 to Ex12, where the propellant includes HFA-152a.

Example Ex15. The inhaler of any one of Ex1 to Ex14, where the metering valve includes a start of life leak rate of no greater than 650 mg/yr.

Example Ex16. A metered dose inhaler that includes a container including a reservoir that contains a formulation that includes a propellant and at least one active pharmaceutical ingredient, where the propellant includes at least one of HEA or HFO; and a metering valve connected to the container. The metering valve includes a valve housing having a first end and a second end disposed in the reservoir, where the valve housing defines a metering chamber between the first end and the second end of the valve housing, and where the metering chamber is adapted to receive the formulation from the reservoir. The metering valve further includes a metering valve stem that extends through the first end of the valve housing, metering chamber, and second end of the valve housing; a diaphragm disposed at least partially within the first end of the valve housing and including an opening, where a first portion of the metering valve stem is disposed through the diaphragm opening and forms a dynamic seal with the diaphragm; and a tank seal disposed at least partially within the second end of the valve housing and including an opening, where a second portion of the metering valve stem is disposed through the tank seal opening and forms a dynamic seal with the tank seal. At least one of the diaphragm or tank seal consists essentially of polytetrafluoroethylene (PTFE). The metering valve further includes a primed position where the formulation can flow between the reservoir and the metering chamber, and where the diaphragm is adapted to seal the metering chamber from an outside atmosphere. The metering valve further includes an actuated position where the second portion of the metering valve stem seals against the tank seal and the second end of the valve housing to seal the metering chamber from the reservoir, thereby defining a metered volume of formulation within the metering chamber, and where the metering valve stem includes an exit chamber allowing the formulation to flow between the metering chamber and the outside atmosphere.

Example Ex17. The inhaler of Ex16, where the diaphragm does not include a spring.

Example Ex18. The inhaler of any one of Ex16 to Ex17, where a pressure within the reservoir is no greater than 20 bar.

Example Ex19. The inhaler of any one of Ex16 to Ex18, where at least one of the diaphragm or tank seal includes a Shore D hardness of 50 to 60.

Example Ex20. The inhaler of any one of Ex16 to Ex19, where a volume of the metering chamber is about 25 to 100 microliters.

Example Ex21. The inhaler of any one of Ex16 to Ex20, further including an actuator having an actuator housing adapted to enclose at least a portion of the metering valve and container.

Example Ex22. The inhaler of Ex21, further including a ferrule that adapted to connect to the metering valve to an end of the container, and a gasket disposed between the ferrule and the first end of the container.

Example Ex23. The inhaler of Ex22, where the gasket consists essentially of PTFE.

Example Ex24. The inhaler of any one of Ex22 to Ex23, further including an O-ring disposed between the ferrule and a side surface of the cannister, where the O-ring consists essentially of PTFE.

Example Ex25. The inhaler of any one of Ex16 to Ex24, where each of the diaphragm and tank seal consists essentially of PTFE.

Example Ex26. The inhaler of any one of Ex16 to Ex25, where the propellant includes HFO-1234ze(E).

Example Ex27. The inhaler of any one of Ex16 to Ex25, where the propellant includes HFA-152a.

Example Ex28. The inhaler of any one of Ex16 to Ex27, where the metering valve includes a start of life leak rate of no greater than 650 mg/yr.

Example Ex29. A method that includes disposing a formulation that includes a propellant and at least one active pharmaceutical ingredient within a reservoir of a container, where the propellant includes at least one of hydrofluoroalkane (HFA) or hydrofluoroolefin (HFO); connecting a metering valve to the container; and sealing at least one interface in the valve or between the container and the valve with a PTFE seal.

Example Ex30. The method of Ex29, where the formulation is disposed within the container prior to connecting the metering valve to the container.

Example Ex31. The method of Ex29, where the formulation is disposed within the container after connecting the metering valve to the container.

Example Ex32. The method of any one of Ex29 to Ex31, where connecting the metering valve to the container includes disposing a valve stem of the metering valve through an aperture in a ferrule and connecting the ferrule to an end of the container such that the metering valve is disposed at least partially within the container.

Example Ex33. The method of Ex32, where sealing at least one interface includes sealing an interface between the end of the container and the ferrule with a PTFE gasket.

Example Ex34. The method of Ex33, where connecting the metering valve to the container further includes disposing a diaphragm within the ferrule such that an opening of the diaphragm is aligned with the aperture of the ferrule and the valve stem is disposed through the opening of the diaphragm and the aperture of the ferrule.

Example Ex35. The method of Ex34, where sealing the at least one interface includes sealing an interface between the metering valve stem and the diaphragm to form a dynamic seal between the metering valve stem and the diaphragm.

Example Ex36. The method of any one of Ex32 to Ex35, where sealing the at least one interface includes sealing an interface between a metering chamber of the metering valve and the reservoir with a tank seal to form a dynamic seal between the metering valve stem and the tank seal, where a portion of the valve stem is disposed through an opening of the tank seal.

All references and publications cited herein are expressly incorporated herein by reference in their entirety into this disclosure, except to the extent they may directly contradict this disclosure. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below.

1. A metered dose inhaler comprising:
a container comprising a reservoir that contains a formulation comprising a propellant and at least one active pharmaceutical ingredient, wherein the propellant comprises at least one of hydrofluoroalkane (HFA) or hydrofluoroolefin (HFO); and
a metering valve connected to the container, the metering valve comprising:
a valve housing comprising a first end and a second end, wherein the valve housing defines a metering chamber between the first end and the second end of the housing, wherein the metering chamber is adapted to receive formulation from the reservoir;
a metering valve stem that extends through the first end of the valve housing, metering chamber, and second end of the valve housing;
a diaphragm disposed at least partially within the first end of the valve housing and comprising an opening, wherein a first portion of the metering valve stem is disposed through the diaphragm opening and forms a dynamic seal with the diaphragm; and
a tank seal disposed at least partially within the second end of the valve housing and comprising an opening, wherein a second portion of the metering valve stem is disposed through the tank seal opening and forms a dynamic seal with the tank seal;
wherein at least one of the diaphragm or tank seal consists essentially of polytetrafluoroethylene (PTFE).

2. The inhaler of claim 1, wherein the diaphragm does not include a spring.

3. The inhaler of claim 1, wherein at least one of the diaphragm or tank seal comprises a Shore D hardness of 50 to 60.

4. The inhaler of claim 1, further comprising an actuator comprising an actuator housing adapted to enclose at least a portion of the metering valve and container.

5. The inhaler of claim 4, further comprising:
a ferrule that adapted to connect to the metering valve to an end of the container; and
a gasket disposed between the ferrule and the first end of the container, wherein the gasket consists essentially of PTFE.

6. The inhaler of claim 5, further comprising an O-ring disposed between the ferrule and a side surface of the container, wherein the O-ring consists essentially of PTFE.

7. The inhaler of claim 1, wherein each of the diaphragm and tank seal consists essentially of PTFE.

8. The inhaler of claim 1, wherein the propellant comprises HFO-1234ze(E).

9. The inhaler of claim 1, wherein the propellant comprises HFA-152a.

10. A metered dose inhaler comprising:
a container comprising a reservoir that contains a formulation comprising a propellant and at least one active pharmaceutical ingredient, wherein the propellant comprises at least one of HFA or HFO; and
a metering valve connected to the container, the metering valve comprising:
a valve housing comprising a first end and a second end disposed in the reservoir, wherein the valve housing defines a metering chamber between the first end and the second end of the valve housing, wherein the metering chamber is adapted to receive the formulation from the reservoir;
a metering valve stem that extends through the first end of the valve housing, metering chamber, and second end of the valve housing;
a diaphragm disposed at least partially within the first end of the valve housing and comprising an opening, wherein a first portion of the metering valve stem is disposed through the diaphragm opening and forms a dynamic seal with the diaphragm; and
a tank seal disposed at least partially within the second end of the valve housing and comprising an opening, wherein a second portion of the metering valve stem is disposed through the tank seal opening and forms a dynamic seal with the tank seal;
wherein at least one of the diaphragm or tank seal consists essentially of polytetrafluoroethylene (PTFE),
wherein the metering valve further comprises a primed position wherein the formulation can flow between the reservoir and the metering chamber, and wherein the diaphragm is adapted to seal the metering chamber from an outside atmosphere; and
wherein the metering valve further comprises an actuated position wherein the second portion of the metering valve stem seals against the tank seal and the second end of the valve housing to seal the metering chamber from the reservoir, thereby defining a metered volume of formulation within the metering chamber, and wherein the metering valve stem comprises an exit chamber allowing the formulation to flow between the metering chamber and the outside atmosphere.

11. The inhaler of claim 10, wherein the diaphragm does not include a spring.

12. The inhaler of claim 10, wherein at least one of the diaphragm or tank seal comprises a Shore D hardness of 45 to 80.

13. The inhaler of claim 10, further comprising an actuator comprising an actuator housing adapted to enclose at least a portion of the metering valve and container.

14. The inhaler of claim 13, further comprising:
a ferrule that is adapted to connect to the metering valve to an end of the container; and
a gasket disposed between the ferrule and the first end of the container, wherein the gasket consists essentially of PTFE.

15. The inhaler of claim 14, further comprising an O-ring disposed between the ferrule and a side surface of the container, wherein the O-ring consists essentially of PTFE.

16. The inhaler of claim 10, wherein each of the diaphragm and tank seal consist essentially of PTFE.

17. The inhaler of claim 10, wherein the propellant comprises HFO-1234ze(E) or HFA-152a.

18. A method comprising:
disposing a formulation comprising a propellant and at least one active pharmaceutical ingredient within a reservoir of a container, wherein the propellant comprises at least one of hydrofluoroalkane (HFA) or hydrofluoroolefin (HFO);
connecting a metering valve to the container; and
sealing at least one interface in the valve or between the container and the valve with a PTFE seal.

19. The method of claim 18, wherein connecting the metering valve to the container comprises:
disposing a valve stem of the metering valve through an aperture in a ferrule; and
connecting the ferrule to an end of the container such that the metering valve is disposed at least partially within the container.

20. The method of claim 19, wherein sealing at least one interface comprises sealing an interface between the end of the container and the ferrule with a PTFE gasket.
